(19) United States
(12) Patent Application Publication
(10) Pub. No.: US 2013/0123468 A1
(43) Pub. Date: May 16, 2013

(54) METHOD FOR EXTRACTING UNDENATURED TYPE II COLLAGEN HAVING ACTIVE EPITOPE

(71) Applicants: Hiroyoshi Moriyama, Tokyo (JP); Orie Yoshinari, Saitama (JP); Yoshiaki Shiojima, Saitama (JP)

(72) Inventors: Hiroyoshi Moriyama, Tokyo (JP); Orie Yoshinari, Saitama (JP); Yoshiaki Shiojima, Saitama (JP)

(21) Appl. No.: 13/812,280

(22) PCT Filed: Jul. 14, 2011
(86) PCT No.: PCT/JP2011/066038

§ 371 Date: Jan. 25, 2013

(30) Foreign Application Priority Data
Sep. 13, 2010 (JP) 2010-204731

(51) Int. Cl. C07K 1/36

(52) U.S. Cl. CPC C07K 1/36
USPC 530/356; 435/273

(57) ABSTRACT

A method for extracting water-soluble undenatured type II collagen having an active epitope comprises a first step for extracting animal-derived cartilage using an acidic solution at 50° C. or less, and a second step for adding pepsin to the acidic solution and performing extraction at 40° C. or less. This allows the epitope of undenatured type II collagen to be extracted in large quantities and with high efficiency without any loss of activity, and makes it possible to provide a food or beverage product obtained using the water-soluble undenatured type II collagen extracted by the extraction method.

### BACKGROUND OF THE INVENTION

The present invention relates to a method for extracting undenatured type II collagen while an activity of its epitope is intact. The present invention also relates to undenatured type II collagen which is extracted by this method and has the epitope whose activity is intact.

### BRIEF SUMMARY OF THE INVENTION

Collagen is a protein that composes skin, bone, cartilage, and the like, and a major component of extracellular matrices of multicellular animals. There are plurality of types of collagen. Among them, type II collagen that is fibrous collagen composed of three chains is mainly contained in articular cartilage and additionally contained in ocular vitreous body. Thus, usefulness of the type II collagen is noticed as a structural functional nutrient factor for the cartilage. Those processed after being treated with high heat or chemically treated are known. Methods for extraction thereof are also disclosed (see Patent Literature 1).

However, in recent researches, it has been found that a three chain structure of collagen is broken down by treatment with high heat or chemical treatment and thus it is effective for chronic rheumatoid arthritis and arthritis to orally administer undeformed type II collagen which does not undergo such a deformation and in which the three chain structure is intact (see Non Patent Literature 1).

On this point, it has been found that immune cells, particularly lymphocytes become immunologically tolerant by ingesting the undenatured type II collagen, and recognize collagen as not a foreign material but an autologous material, thereby not attacking endogenous collagen. Thus currently, undeformed type II collagen in which the three chain structure is intact is known to be obtained by washing, drying and pulverizing animal-derived cartilage without treating with high heat, and is known to be used.

Further, by the recent research, it has been found that the presence of the epitope is indispensable for inducing the immune tolerance. The epitope is a specific structural unit of an antigen, which is recognized and bound by an antibody. When the antibody is bound to a pathogenic microorganism or a macromolecular substance, the antibody does not recognize the entire organism or substance but recognizes the epitope thereon and binds to it. The epitope is a minimum unit for antigenicity and is also referred to as an antigenic determinant. It is believed that the active epitope is bound to Peyer's patch present as a tissue in small intestine to activate immune (lymph) cells, and information transmitting molecules such as cytokines and chemokines involved in proliferation, differentiation, and suppression of other cells are secreted to suppress inflammation such as arthritis (see Non Patent Literature 2).

Therefore, even if the collagen is the undeformed type II collagen, unless the activity of the epitope is intact, the collagen can not be recognized by the antibody, can not induce the immune tolerance and reduces its usefulness.

Also in Non Patent Literature 2, it is suggested that the active epitope in a larger amount can be extracted when the type II collagen is extracted with an acid solution containing pepsin in consideration of in vivo reaction. This is speculated to be because the addition of the acidic solution containing pepsin affects a shape of the collagen, e.g., a three-dimensional shape of the three chain structure in the collagen is loosened, and consequently an amount of the exposed epitope is increased.

Thus in this application, type II collagen in which the three-dimensional shape of the three chain structure is deformed (e.g., loosened) and the activity of the epitope is intact is referred to as “undenatured type II collagen”, while type II collagen in which the three-dimensional shape of the three chain structure remains intact is referred to as “undeformed type II collagen”. They are distinguished in terms.

On the contrary in Patent Literature 1, the epitope is not focused at all, and collagen is extracted with hot water (treatment with high heat), which destroys the three chain structure of type II collagen, in Example thereof (Example 2). No specific method for efficient extraction is investigated therein.

Meanwhile, the aforementioned undeformed type II collagen in which the three chain structure is intact is only suspended and not dissolved in water even when pulverized into considerably small particles, and is limited for applying to beverages and liquid foods.

Further, the collagen is necessary to be ingested continuously in order to be useful for articular diseases such as chronic rheumatoid arthritis and knee osteoarthritis. Cost cutting and efficiency of production are strongly required under current economic circumstance. Food forms are also needed in consideration of quality of life of the increasing elderly.

Therefore, if a method capable of efficiently extracting the type II collagen in a large amount with keeping the activity of the epitope intact is available, more than comparable effects can be obtained even though a raw material and a necessary ingestion dose are required in less than conventional amounts. Further, if the collagen is soluble in water, it can be easily ingested orally.

### CITATION LIST

### Patent Literature

Patent Literature 1: Japanese Patent No. 3155746

### Non Patent Literatures

Non Patent Literature 1: Trenthamm, D. E. et al., Autoimmunity to type II collagen: an experiment model of arthritis. J. Exp. Med., 1977, 146, 857-867.

Non Patent Literature 2: Bagchi, D. et al., Effects of orally administered undenatured type II collagen against arthritic inflammatory diseases: a mechanistic exploration. Int. J. Clin. Pharm. Res., 2002, 22 (3/4): 101-10.

### DETAILED DESCRIPTION

### Technical Problem

Thus, it is an object of the present invention to provide a method capable of efficiently extracting an epitope of undenatured type II collagen in a large amount with keeping an activity of the epitope intact.

It is another object of the present invention to provide a food or beverage product obtained using water-soluble undenatured type II collagen extracted by the extraction method.

### Solution to Problem

As a result of an extensive study for solving the above problem, the present inventors have found that an amount of an active epitope capable of being extracted is not large though the water-soluble undeformed type II collagen can be extracted with an acidic solution alone.

Nevertheless, the present inventors have found by a further extensive study that not only the active epitope can be extracted in a larger amount but also an extraction time period can be shortened more efficiently than in the case of extracting once with the acidic solution containing pepsin, by dividing an extraction process into two stages of a first extraction with the acidic solution and a second extraction with the acidic solution containing pepsin.

This can simultaneously accomplish all of increase of the epitope, keeping the activity intact, efficiency of extraction, and water solubilization, and the present invention has been completed.

That is, the method for extracting the water-soluble undenatured type II collagen having the active epitope of the present invention is composed of a first extraction step of extracting animal-derived cartilage with the acidic solution at 50° C. or below and a second extraction step of adding pepsin to the acidic solution and performing extraction at 40° C. or below.

In a condition for the first extraction step, temperature, PH, and the extraction time period are suitably 5 to 35° C. (preferably 25° C.), pH 3.4 or less, and 12 to 36 hours, respectively.

In a condition for the second extraction step, the temperature and the extraction time period are suitably 5 to 35° C. and 12 to 36 hours, respectively.

A sum of the extraction time periods in the first and second steps is suitably 48 hours or less.

It is also suitable to perform the second extraction at lower temperature than in the first extraction.

It is also suitable that elastase is further added in the second extraction step.

It is also suitable that the acidic solution is an acetic acid solution or a citric acid solution.

It is also suitable that the animal-derived cartilage is chicken cartilage, which is washed and dried followed by being pulverized.

It is further suitable to be the undenatured type II collagen having the active epitope obtained by the above method and the food or beverage product containing the same.

In this application, the “active epitope” refers to an epitope that can be recognized by an antibody to induce the immune tolerance. The epitope of the type II collagen is sometimes different depending on the animal from which the collagen is derived, and for example, in the case of cattle, the epitope is represented by the following structural formula.

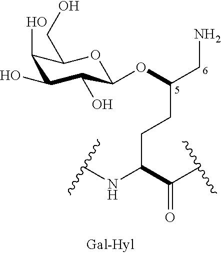

Types of the animal that can be employed in the present invention include, but are not limited to birds, mammals, and fish such as chicken, cattle, swine, and the like. The type II collagen is contained mainly in the cartilage and also additionally in nasal cartilage in salmon.

The animal-derived cartilage is typically washed and dried as a pretreatment before being pulverized for the extraction. Known washing solutions such as a hydrogen peroxide solution and a sodium hypochlorite solution can be used for washing, and the washing is typically performed at 40° C. or below for about 10 to 30 minutes. Known drying methods such as freeze drying (−40 to −10° C.) and oven drying (40 to 50° C.) can be used for drying. When dried at high temperature of 60° C. or above, the type II collagen and the active epitope are potentially denatured to reduce their yields. Thus, this is not suitable. The pulverization can be performed using a known pulverizer, and it is preferable to pulverize to 5 mm or smaller and preferably 3 mm or smaller particles. This is because the smaller a particle size is, the more the efficiency of the extraction is increased.

Elastase is a protease (proteolytic enzyme) that cleaves a protein and peptone into smaller polypeptides and amino acids. By the use of elastase in combination with pepsin, the type II collagen is degraded or cleaved into smaller peptides. Thus, the combination thereof is preferable because the active epitope is further exposed.

In the present invention, the extraction process is composed of two stages of the first extraction with the acidic solution and the second extraction with the acidic solution containing pepsin. The extraction time period for the first extraction and the second extraction may be appropriately determined depending on a concentration of an acid, an extraction temperature, and an amount of the active epitope to be extracted. In order to enhance the efficiency, it is suitable to perform each of the first extraction and the second extraction for 12 hours or more and preferably about 24 hours. Such a constitution can make the extraction time period at least 30% more efficient than in the case of performing the extraction with the acidic solution containing pepsin at a time.

The first extraction is performed at a temperature of 50° C. or below and preferably about 5 to 35° C. that is an ambient temperature so that the structure of the type II collagen is not broken down. In particular, the extraction at about room temperature (about 25° C.) makes the efficiency high.

The second extraction is performed at a temperature of 40° C. or below and preferably about 5 to 35° C. that is an ambient temperature. To avoid the denaturation of collagen that is a protein as much as possible, the temperature may be set to 5° C. to temperature that is lower than that in the first extraction.

The undenatured type II collagen having the active epitope and extracted by the method of the present invention can be used in the form of an aqueous solution itself or in the form of powder obtained by performing centrifugation or filtration followed by oven drying or freeze drying. The obtained powder is soluble in water. Therefore, they can easily be contained in the food or beverage product.

### Advantageous Effects of Invention

According to the present invention, it is possible to not only extract the undenatured type II collagen with keeping the activity of the epitope intact but also increase the active epitope capable of being recognized by the antibody compared with the undeformed type II collagen in which the three-dimensional shape of the three chain structure remains intact. Further, since the extraction time period can be shortened dramatically and the obtained collagen is soluble in water, it is suitable for industrial mass production, and can provide a material in various forms applicable to the food and the beverage products.

### DESCRIPTION OF EMBODIMENTS

The method for extracting the active epitope of the present invention was verified in various points.

An ELISA method (“native Type II Collagen Capture ELISA” supplied from Astarte Biologics) was used for quantifying the active epitope of the type II collagen. In this ELISA method, an amount of the type II collagen reacted with the active epitope can be figured out using an antibody antigen reaction, and consequently the amounts of the active epitope can be compared. Pepsin used in the following experiments was manufactured by Wako Pure Chemical Industries Ltd. and was derived from swine gastric mucosa, and elastase used was manufactured by Wako Pure Chemical Industries Ltd. and was derived from swine pancreas.

### Experimental Example 1

### Examination of Drying Method and Presence or Absence of Enzyme

Experiments were carried out in order to examine whether an amount of the finally obtained active epitope was different depending on difference of a drying method of chicken sternum cartilage and whether an extraction efficiency was different depending on the presence or absence of an enzyme.

Steps are as follows. Washed chicken breast cartilage was freeze-dried or dried in an oven, subsequently pulverized, then extracted with one of three solutions, i.e., an acetic acid solution, or the acetic acid solution plus pepsin, or the acetic acid solution plus pepsin plus elastase at 5° C. at pH 3 for 72 hours (concentration of pepsin was 1 mg/ml, the same shall apply to the following experiments), followed by centrifugation (10,000×g, 20 minutes, 4° C., the same shall apply to the following experiments), and then an amount of the type II collagen reacted with the active epitope was quantified by the above ELISA method (% by weight, the same shall apply to the following experiments). The results are shown in table 1.

TABLE 1
Steps: Freeze drying or oven drying → with or without pepsin for
72 hours → with or without elastase overnight → supernatant from
centrifugation (Acetic acid, pH 3, 5° C.)

| Drying treatment | Enzyme | Content in cartilage (% w/w) |
|---|---|---|
| Freeze drying | None | 2.74 |
| (8 h) | Pepsin | 16.4 |
| | Pepsin + Elastase | 23.1 |
| Oven drying | None | 3.19 |
| (45 °C., 8 h) | Pepsin | 17.2 |
| | Pepsin + Elastase | 25.8 |

From the above results, it was found that the amount of the active epitope was scarcely different in the freeze drying and oven drying. Thus, it is conceivable that the oven drying is more suitable for mass processing in terms of easiness and cost of the method (unless otherwise specified, the chicken sternum cartilage was washed, dried in the oven and pulverized in the following experiments).

It was also found that values obtained by the treatment with the acid solution alone were low, values were higher when pepsin was added and values were much higher when pepsin and elastase were added.

This is speculated to be because the three-dimensional structure of the three chains is loosened by the addition of pepsin and is further cleaved into smaller peptides to expose the active epitope more abundantly by the addition of elastase.

That is, the type II collagen having the active epitope in a larger amount than that in the undeformed type II collagen obtained by washing, drying and pulverizing the animal-derived cartilage is present in the extracted solution obtained by extracting with the acid solution containing pepsin. Thus, it is possible to obtain higher effects with the raw material in a less amount. Further, this undenatured type II collagen is dissolved in the acidic solution, and thus is highly applicable as the undenatured type II collagen soluble in water.

### Experimental Example 2

### Difference of Effect Due to Type of Acid in Extraction

The type of the acid to be used for the extraction was changed, and pepsin and elastase were added thereto. Using these extraction solutions, the extraction was carried out at 5° C. at pH 3 for 72 hours. Both the freeze drying and the oven drying were examined for acetic acid and citric acid. The results are shown in Table 2.

TABLE 2
Steps: Freeze drying or oven drying → with or without
pepsin for 72 hours → elastase reaction overnight →
supernatant from centrifugation

| Dry | Acid type | | | Content in |
|---|---|---|---|---|
| treatment | (pH 3, 5° C.) | pH | Enzyme | cartilage (% w/w) |
| Freeze | Acetic acid | 3 | Pepsin + | 23.1 |
| drying | Citric acid | | Elastase | 23.9 |
| Oven | Acetic acid | | (Reaction | 25.8 |
| Drying | Citric acid | | 5° C.) | 26.2 |
| (45° C., | Nitric acid | | | 24.5 |
| 8h) | Phosphoric acid | | | 24.9 |
| | Hydrochloric acid | | | 18.6 |
| | Sulfuric acid | | | 23.3 |
| | Malic acid | | | 22.3 |

From the above results, some differences were observed depending on the acid used for the extraction. In particular, the acetic acid or the citric acid is suitable in consideration of circumstances such as possibility of application to foods and beverages, availability and prices. Extracted amounts at a time point after 24 hours had passed and a time point after 48 hours had passed were about 30% and about 70% relative to the amount at a time point after 72 hours had passed, respectively.

### Experimental Example 3

### Presence or Absence of Treatment with High Heat after Extraction

The type II collagen was extracted under the same condition, treated with high heat (100° C., 30 minutes) or not treated with heat, and then quantified. The results are shown in following Table 3.

TABLE 3
Steps: Oven drying → pepsin reaction for 72 hours →
elastase reaction overnight → supernatant from
centrifugation → heated

| | | | | Treatment | Content in |
|---|---|---|---|---|---|
| Dry | Acid type | | | with high heat | cartilage |
| treatment | (pH 3, 5° C.) | pH | Enzyme | (100° C. 30 min) | (% w/w) |
| Freeze | Acetic acid | 3 | Pepsin + | Presence | 4.31 |
| Drying | Citric acid | | Elastase | | 5.23 |
| | Acetic acid | | (Reaction | Absence | 17.0 |
| | Citric acid | | 5° C.) | | 20.4 |

From the above results, it was found that the activity of the epitope was lost by treating with high heat. Thus, it is conceivable to be suitable that the extraction is also carried out at least at 50° C. or below.

### Experimental Example 4

### Difference of Extraction Effect Due to pH

The amount of the active epitope was quantified by changing a pH value in the acetic acid solution and the citric acid solution and not changing the other conditions. The results are shown in following Table 4.

TABLE 4
Steps: Oven drying → with or without pepsin for
72 hours → elastase reaction overnight →
supernatant from centrifugation

| | | | | Content in |
|---|---|---|---|---|
| Dry | Acid type | | | cartilage |
| treatment | (5° C.) | pH | Enzyme | (% w/w) |
| Oven | Acetic | 2.0 | Pepsin + | 26.2 |
| Drying | acid | 2.5 | Elastase | 24.1 |
| | | 3.0 | (Reaction | 23.2 |
| | | 3.4 | 5° C.) | 19.4 |
| | Citric | 2.0 | | 27.0 |
| | acid | 2.5 | | 25.8 |
| | | 3.0 | | 24.0 |
| | | 3.4 | | 21.3 |

From the above results, it was found that the lower the pH value, the higher the obtained value. Meanwhile, it was found that a certain amount could be extracted when the pH value is around 3.4. An excessively strong acid causes problems in maintenance and durability of containers and production equipments. Thus, the pH value of about 2.5 to 3.0 is most suitable in consideration of total efficiency including economic efficiency.

### Experimental Example 5

### Effect of Two Stage Extraction According to the Present Invention, and Relation of Extraction Temperature Therewith

The amount of the active epitope was quantified by performing the first extraction for 24 hours and the second extraction for 24 hours and changing the temperature in the first extraction. The results are shown in Table 5.

TABLE 5
Steps: Acid extraction for 24 hours → pepsin reaction
for 24 hours → supernatant from centrifugation

| | | | | Content in |
|---|---|---|---|---|
| | Extraction | | | cartilage |
| Acid type | temperature (° C.) | pH | Enzyme | (% w/w) |
| Acetic | 5 | 3 | Pepsin | 16.4 |
| Acid | Room temperature* | | (Reaction | 20.3 |
| | 40 | | 5° C.) | 18.9 |
| | 60 | | | 7.7 |
| | 100 | | | 2.7 |
| Citric | 5 | | | 15.5 |
| Acid | Room temperature* | | | 19.2 |
| | 40 | | | 20.9 |
| | 60 | | | 13.1 |
| | 100 | | | 4.7 |

*about 25° C.

From the above results, the extracted amount that was 20.3% compared with the case of extracting with only the acid solution containing pepsin for 72 hours (see 17.2% in Experimental Example 1) could be realized in only 48 hours. This dramatically enhanced the extraction efficiency, and the undenatured type II collagen having the active epitope in a large amount could be extracted from the raw material in a small amount in a short period of time. The solution of the extracted undenatured type II collagen having the active epitope is soluble in water, and is more easily applied to the foods and beverages.

The first extraction has no problem at 40° C., but when the first extraction is performed at 60° C. or above, the extracted amount is obviously reduced. Thus, the extraction temperature should be 50° C. or below, and is suitably the ambient temperature (about 15 to 35° C., in particular, about 25° C. is preferable). The second extraction was performed at 5° C. in order to avoid the denaturation of collagen as possible, but when the second extraction is performed at ambient temperature or temperature that is higher than 5° C., pepsin is potentially further activated and the extraction efficiency is potentially increased, indicating the high effect of the present invention.

Further, as is evident from the aforementioned experiments, it is possible to further increase the extraction efficiency by adding elastase into the second extraction.

From the above experiment results, it has been found that the active epitope in a large amount can be extracted and the water-soluble and highly applicable type II collagen can be obtained in a short period of time from the raw material in a small amount according to the method of the present invention. Thus, according to the present invention, it has been found that the method of the present invention is excellent in all of extraction time period, extraction efficiency, and applicability of the extract compared to conventional methods.

1-11. (canceled)

12. An extraction method wherein water-soluble undenatured type II collagen is extracted and an active epitope contained in the water-soluble undenatured type II collagen is extracted, comprising the steps of:
extracting animal-derived cartilage with an acidic solution under the following conditions:
(1) temperature: 5 to 35° C.,
(2) pH: 3.4 or less, and
(3) extraction time period: 12 to 36 hours; and
adding pepsin to the acidic solution and extracting an active epitope with the acidic solution under the following conditions:
(1) temperature: 5 to 35° C.,
(2) pH: 3.4 or less, and
(3) extraction time period: 12 to 36 hours.

13. The extraction method according to claim 12, wherein the step of extracting animal-derived cartilage with the acidic solution is a first step and the step of adding pepsin to the acidic solution and extracting an active epitope with the acidic solution is a second step, and the total extraction time for both the first step and the second step is 48 hours or less.

14. The extraction method according to claim 13, wherein the second step is performed at lower temperature than the first step.

15. The extraction method according to claim 13, wherein elastase is further added in the second step of extraction.

16. The extraction method according to claim 14, wherein elastase is further added in the second step of extraction.

17. The extraction method according to claim 12, wherein the acidic solution is an acetic acid solution or a citric acid solution.

18. The extraction method according to claim 13, wherein the acidic solution is an acetic acid solution or a citric acid solution.

19. The extraction method according to claim 14, wherein the acidic solution is an acetic acid solution or a citric acid solution.

20. The extraction method according to claim 15, wherein the acidic solution is an acetic acid solution or a citric acid solution.

21. The extraction method according to claim 12, wherein the animal-derived cartilage is washed and dried at 60° C. or lower followed by being pulverized.

22. The extraction method according to claim 13, wherein the animal-derived cartilage is washed and dried at 60° C. or lower followed by being pulverized.

23. The extraction method according to claim 14, wherein the animal-derived cartilage is washed and dried at 60° C. or lower followed by being pulverized.

24. The extraction method according to claim 12, wherein the animal-derived cartilage is chicken cartilage.

25. The extraction method according to claim 13, wherein the animal-derived cartilage is chicken cartilage.

26. The extraction method according to claim 14, wherein the animal-derived cartilage is chicken cartilage.

27. The extraction method according to claim 15, wherein the animal-derived cartilage is chicken cartilage.

28. The extraction method according to claim 17, wherein the animal-derived cartilage is chicken cartilage.

29. The extraction method according to claim 21, wherein the animal-derived cartilage is chicken cartilage.

30. An undenatured type II collagen having an active epitope, produced by:
extracting animal-derived cartilage with an acidic solution under the following conditions:
(1) temperature: 5 to 35° C.,
(2) pH: 3.4 or less, and
(3) extraction time period: 12 to 36 hours, and
adding pepsin to the acidic solution and extracting an active epitope with the acidic solution under the following conditions:
(1) temperature: 5 to 35° C.,
(2) pH: 3.4 or less, and
(3) extraction time period: 12 to 36 hours.

31. A food or beverage product containing the undenatured type II collagen having the active epitope, produced by:
extracting animal-derived cartilage with an acidic solution under the following conditions:
(1) temperature: 5 to 35° C.,
(2) pH: 3.4 or less, and
(3) extraction time period: 12 to 36 hours, and
adding pepsin to the acidic solution and extracting an active epitope with the acidic solution under the following conditions:
(1) temperature: 5 to 35° C.,
(2) pH: 3.4 or less, and
(3) extraction time period: 12 to 36 hours.
